(19) United States
(12) Patent Application Publication
(10) Pub. No.: US 2008/0281163 A1
(43) Pub. Date: Nov. 13, 2008

(54) APPARATUS AND METHOD FOR ACQUIRING MEDICAL DATA

(75) Inventors: James R. Peterson, Fond du Lac, WI (US); Sarah Alme, Milwaukee, WI (US); Mark R. Kohls, New Berlin, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/746,968

(22) Filed: May 10, 2007

(51) Int. Cl. A61B 5/00

(52) U.S. Cl. 600/300

(57) ABSTRACT

A medical diagnostic/monitoring system is disclosed herein. The medical diagnostic/monitoring system includes a data collection system having a sensor, and a collection device connected to the sensor. The collection device includes a first near field communication device. The medical diagnostic/monitoring system also includes a signal acquisition device wirelessly connected to the data collection system. The signal acquisition device includes a second near field communication device. The first near field communication device and the second near field communication device are collectively configured to wirelessly transfer data from the data collection system to the signal acquisition device.

### FIELD OF THE INVENTION

This disclosure relates generally to an apparatus and method for acquiring medical data such as, for example, ECG data or EEG data.

### BACKGROUND OF THE INVENTION

An electrocardiogram (ECG) is a noninvasive test adapted to assess a patient's heart condition. The ECG can be performed by measuring electrical potential between various locations on the patient's body. The electrical potential measurements are obtained with a plurality of electrodes secured directly to the patient. The plurality of electrodes are operatively connected to a corresponding plurality of lead wires that are typically physically connected to a signal acquisition device. The physical connection between the plurality of lead wires and the signal acquisition device is generally formed with one or more electrical connectors.

One problem with conventional ECG systems that implement electrical connectors to physically connect the lead wires with the signal acquisition device is that the electrical connectors are expensive. The expense associated with the electrical connectors adds to the overall cost of the ECG system and also makes it more difficult to economically justify providing systems incorporating disposable leads. Therefore, the leads are generally re-used on multiple patients and must be cleaned and sterilized after each usage. The process of cleaning and sterilizing the leads is costly and time consuming.

Another problem with conventional ECG systems that implement electrical connectors to physically connect the lead wires with the signal acquisition device is that the electrical connectors are heavy. The added weight can pull on the electrodes attached to the patient which may cause discomfort. Additionally, the weight of some conventional electrical connectors can detach the electrodes from the patient or otherwise decouple the patient from the signal acquisition device.

Another problem with conventional ECG systems that implement electrical connectors to physically connect the lead wires with the signal acquisition device is that the electrical connectors can become unreliable over time. An unreliable electrical connector can interrupt the signal transferred to the signal acquisition device such that the patient cannot be monitored and/or the resultant ECG data is imprecise.

Electroencephalogram (EEG) systems incorporate a plurality of electrodes that are applied to a patient's head in order to assess brain function. EEG systems generally implement electrical connectors to physically couple the plurality of electrodes with a signal acquisition device in a manner similar to that of ECG systems. EEG systems therefore share many of the problems previously described with respect to ECG systems.

### BRIEF DESCRIPTION OF THE INVENTION

The above-mentioned shortcomings, disadvantages and problems are addressed herein which will be understood by reading and understanding the following specification.

In an embodiment, a medical diagnostic/monitoring system includes a data collection system having a sensor, and a collection device connected to the sensor. The collection device includes a first near field communication device. The medical diagnostic/monitoring system also includes a signal acquisition device wirelessly connected to the data collection system. The signal acquisition device includes a second near field communication device. The first near field communication device and the second near field communication device are collectively configured to wirelessly transfer data from the data collection system to the signal acquisition device.

In another embodiment, an electrocardiogram system includes a data collection system having a sensor, a conductor connected to the sensor, and a collection device connected to the conductor. The collection device includes a first near field communication device. The collection device is configured to be inexpensive and lightweight. The electrocardiogram system also includes a signal acquisition device wirelessly connected to the data collection system. The signal acquisition device includes a second near field communication device. The first near field communication device and the second near field communication device are collectively configured to wirelessly transfer power from the signal acquisition device to the data collection system and to wirelessly transfer data from the data collection system to the signal acquisition device.

In another embodiment, a method for acquiring electrocardiogram data includes generating an electromagnetic field using a near field communication device, implementing the electromagnetic field to wirelessly transfer power from a signal acquisition device to a data collection system, sensing a cardiac electrical signal with the data collection system, generating an analog signal proportional to the sensed cardiac electrical signal, converting the analog signal into a digital signal, and manipulating the electromagnetic field to wirelessly transfer the digital signal from the data collection system to the signal acquisition device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a patient connected to a medical diagnostic/monitoring system in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

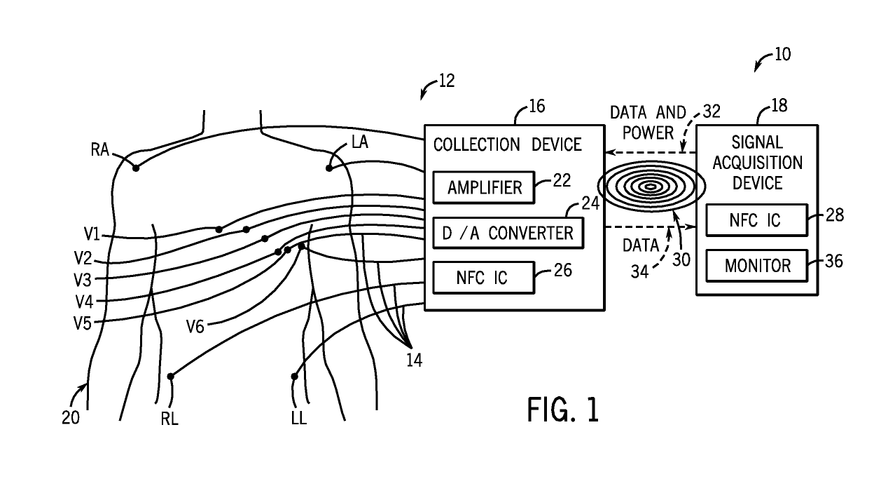

Referring to FIG. 1, a schematically represented medical diagnostic/monitoring system 10 is shown. The medical diagnostic/monitoring system 10 will hereinafter be described as an electrocardiogram (ECG) system 10 adapted measure an electrical signal generated by a patient's heart. It should, however, be appreciated that the medical diagnostic/monitoring system 10 may also include other systems and devices such as, for example, an electroencephalogram (EEG) system, a blood pressure monitor, a pulse oximeter, a thermometer, etc.

The ECG system 10 includes a data collection system 12 and a signal acquisition device 18. For purposes of this disclosure, the data collection system 12 should be defined to include a plurality of sensors or transducers such as the electrodes RA, LA, V1, V2, V3, V4, V5, V6, RL and LL, a plurality of conductors such as the lead wires 14, and a collection device 16.

In the embodiment depicted in FIG. 1, the electrode RA is applied to the patient's right arm; the electrode LA is applied to the patient's left arm; the electrodes V1, V2, V3, V4, V5 and V6 are applied to the patient's chest; the electrode RL is applied to the patient's right leg; and the electrode LL is applied to the patient's left leg. This application of the electrodes provides a standard twelve lead, ten-electrode ECG signal. It should be appreciated that the electrode configuration of FIG. 1 is provided for illustrative purposes, and that other electrode configurations can be envisioned. The electrodes RA, LA, V1, V2, V3, V4, V5, V6, RL and LL are adapted to sense or detect cardiac electrical signals generated by the patient's heart, and to generate analog signals proportional to the detected cardiac electrical signals.

The lead wires 14 are each adapted to couple one of the electrodes RA, LA, V1, V2, V3, V4, V5, V6, RL and LL with the collection device 16. The lead wires 14 are configured to transmit the analog signals from the electrodes RA, LA, V1, V2, V3, V4, V5, V6, RL and LL to the collection device 16.

The collection device 16 is adapted to collect data from the electrodes RA, LA, V1, V2, V3, V4, V5, V6, RL and LL, and to wirelessly transfer the collected data to the signal acquisition device 18 in a convenient format. Advantageously, the collection device 16 can be configured to be both compact and lightweight. According to one embodiment, the collection device 16 can be produced as a 1.5 by 2.5-inch rectangular patch weighing approximately fifteen grams that is adapted for attachment to a patient's chest. As the collection device 16 is lightweight and securable to the patient 20, it is unlikely to pull on the electrodes RA, LA, V1, V2, V3, V4, V5, V6, RL and LL causing patient discomfort or electrode detachment.

The collection device 16 can also be produced inexpensively, which reduces the overall cost of the data collection system 12. Advantageously, the low cost of the collection device 16 enables the production of disposable data collection systems 12. It should be appreciated that it was generally not economically feasible to provide comparable prior art systems that were disposable because the requisite electrical connectors were prohibitively expensive. The data collection system 12 wirelessly transfers data to the signal acquisition system 18 such that the expensive electrical connectors are not required and the overall system cost is reduced. Providing a disposable data collection system 12 eliminates the time and expense associated with cleaning and sterilizing the components after each usage.

According to one embodiment, the collection device 16 includes an amplifier 22, a digital/analog (D/A) converter 24, and a near field communication (NFC) integrated circuit (IC) 26. The amplifier 22 is provided to boost or amplify the relatively weak cardiac electrical activity in a manner adapted to facilitate the process of interpreting the resultant data. This process is well known and will therefore not be described in detail. The D/A converter 24 is configured to convert the analog signals from the electrodes RA, LA, V1, V2, V3, V4, V5, V6, RL and LL into a digital signal that can be transferred to and processed by the signal acquisition device 18.

The NFC IC 26 of the collection device 16 works in combination with a NFC IC 28 of the signal acquisition device 18 in the following manner. The NFC IC 28 produces an electromagnetic field 30 that is operable to wirelessly transmit power and data 32 from the signal acquisition device 18 to the collection device 16, and which can be manipulated by the NFC IC 26 to wirelessly transmit data 34 from the collection device 16 to the signal acquisition device 18. Near field communication is well known to those skilled in the art and therefore will not be described in detail.

The transmission of power 32 from the signal acquisition device 18 to the collection device 16 allows the data collection system 12 to operate without its own dedicated power supply. The elimination of a dedicated power supply allows for a lighter and less expensive data collection system 12. The wireless transmission of data 34 from the collection device 16 to the signal acquisition device 18 allows the data collection system 12 to operate without conventional electrical connectors (not shown) adapted to establish a physical connection between the signal acquisition device 18 and the electrodes RA, LA, V1, V2, V3, V4, V5, V6, RL and LL. The elimination of these conventional electrical connectors further reduces the weight and expense of the data collection system 12.

The signal acquisition device 18 may optionally include a monitor 36. Therefore, after the signal acquisition device 18 receives the data 34 from the collection device 16, the data 34 can be displayed on the monitor 36 and visually analyzed in a conventional manner.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

We claim:

1. A medical diagnostic/monitoring system comprising:
a data collection system comprising:
a sensor; and
a collection device connected to the sensor, said collection device including a first near field communication device; and
a signal acquisition device wirelessly connected to the data collection system, said signal acquisition device including a second near field communication device;
wherein said first near field communication device and said second near field communication device are collectively configured to wirelessly transfer data from the data collection system to the signal acquisition device.

2. The medical diagnostic/monitoring system of claim 1, wherein the medical diagnostic/monitoring system comprises an electrocardiogram system.

3. The medical diagnostic/monitoring system of claim 1, wherein the medical diagnostic/monitoring system comprises an electroencephalogram system.

4. The medical diagnostic/monitoring system of claim 1, wherein the data collection system is disposable.

5. The medical diagnostic/monitoring system of claim 1, wherein the sensor comprises a plurality of electrodes.

6. The medical diagnostic/monitoring system of claim 1, wherein the collection device includes an amplifier configured to amplify signals from the sensor.

7. The medical diagnostic/monitoring system of claim 1, wherein the collection device includes a converter configured to convert analog data from the sensor into digital data.

8. The medical diagnostic/monitoring system of claim 1, wherein the collection device is configured to be inexpensive and lightweight.

9. The medical diagnostic/monitoring system of claim 1, wherein the signal acquisition device includes a monitor.

10. The medical diagnostic/monitoring system of claim 1, wherein the data collection system includes a conductor configured to couple the sensor with the collection device.

11. An electrocardiogram system comprising:
a data collection system comprising:
a sensor;
a conductor connected to the sensor; and
a collection device connected to the conductor, said collection device including a first near field communication device, wherein said collection device is configured to be inexpensive and lightweight; and
a signal acquisition device wirelessly connected to the data collection system, said signal acquisition device including a second near field communication device;
wherein said first near field communication device and said second near field communication device are collectively configured to wirelessly transfer power from the signal acquisition device to the data collection system and to wirelessly transfer data from the data collection system to the signal acquisition device.

12. The electrocardiogram system of claim 11, wherein the data collection system is disposable.

13. The electrocardiogram system of claim 11, wherein the sensor comprises a plurality of electrodes.

14. The electrocardiogram system of claim 11, wherein the conductor comprises a plurality of lead wires.

15. The electrocardiogram system of claim 11, wherein the collection device includes an amplifier configured to amplify signals from the sensor.

16. The electrocardiogram system of claim 11, wherein the collection device includes a converter configured to convert analog data from the sensor into digital data.

17. The electrocardiogram system of claim 11, wherein the signal acquisition device includes a monitor.

18. A method for acquiring electrocardiogram data comprising:
generating an electromagnetic field using a near field communication device;
implementing the electromagnetic field to wirelessly transfer power from a signal acquisition device to a data collection system;
sensing a cardiac electrical signal with the data collection system;
generating an analog signal proportional to the sensed cardiac electrical signal;
converting the analog signal into a digital signal; and
manipulating the electromagnetic field to wirelessly transfer the digital signal from the data collection system to the signal acquisition device.

19. The method of claim 18, further comprising amplifying the cardiac electrical signal.
